SEQUENCE LISTING (45 sequences)

SEQ ID NO: 1 - protein, 11 aa, Artificial Sequence
  CDR-L1
RASQDISKYLN

SEQ ID NO: 2 - protein, 7 aa, Artificial Sequence
  CDR-L2
HTSRLHS

SEQ ID NO: 3 - protein, 9 aa, Artificial Sequence
  CDR-L3
QQGNTLPYT

SEQ ID NO: 4 - protein, 7 aa, Artificial Sequence
  CDR-H1
GVSLPDY

SEQ ID NO: 5 - protein, 5 aa, Artificial Sequence
  CDR-H2
WGSET

SEQ ID NO: 6 - protein, 12 aa, Artificial Sequence
  CDR-H3
HYYYGGSYAMDY

SEQ ID NO: 7 - protein, 107 aa, Artificial Sequence
  fmc63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 120 aa, Artificial Sequence
  fmc63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - protein, 242 aa, Artificial Sequence
  fmc63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 10 - protein, 106 aa, Artificial Sequence
  hVL-1
DIQMTQSPSSLSASVGDRVTITCRASQDISKYLNWYQQKPGKAPKLLLYHTSRLHSGVPSRFSGSGSGTDYTLTISSLQQEDFATYYCQQGNTLPYTFGQGTKVEI

SEQ ID NO: 11 - protein, 120 aa, Artificial Sequence
  hVH-1
QVTLKESGPVLVKPTETLTLTCTVSGVSLPDYGVSWIRQPPGKALEWLAVIWGSETTYYNSALKSRLTISKDTSKSQVVLTMTNMDPVDTATYYCARHYYYGGSYAMDYWGQGTMVTVSS

SEQ ID NO: 12 - protein, 106 aa, Artificial Sequence
  hVL-2
DIQMTQSPSSVSASVGDRVTITCRASQDISKYLNWYQQKPGKAPKLLIYHTSRLHSGVPSRFSGSGSGTDFTLTISSLQPEDFATYYCQQGNTLPYTFGQGTRLEI

SEQ ID NO: 13 - protein, 120 aa, Artificial Sequence
  hVH-2
QVTLKESGPVLVKPTETLTLTCTVSGVSLPDYGVSWIRQPPGKALEWLAVIWGSETTYYNSALKSRLTISKDTSKSQVVLTMTNMDPVDTATYYCAKHYYYGGSYAMDYWGQGTMVTVSS

SEQ ID NO: 14 - protein, 241 aa, Artificial Sequence
  hCD19-1
QVTLKESGPVLVKPTETLTLTCTVSGVSLPDYGVSWIRQPPGKALEWLAVIWGSETTYYNSALKSRLTISKDTSKSQVVLTMTNMDPVDTATYYCARHYYYGGSYAMDYWGQGTMVTVSSGGGGSGGGGSGGGGSDIQMTQSPSSLSASVGDRVTITCRASQDISKYLNWYQQKPGKAPKLLLYHTSRLHSGVPSRFSGSGSGTDYTLTISSLQQEDFATYYCQQGNTLPYTFGQGTKVEI

SEQ ID NO: 15 - protein, 241 aa, Artificial Sequence
  hCD19-2
DIQMTQSPSSLSASVGDRVTITCRASQDISKYLNWYQQKPGKAPKLLLYHTSRLHSGVPSRFSGSGSGTDYTLTISSLQQEDFATYYCQQGNTLPYTFGQGTKVEIGGGGSGGGGSGGGGSQVTLKESGPVLVKPTETLTLTCTVSGVSLPDYGVSWIRQPPGKALEWLAVIWGSETTYYNSALKSRLTISKDTSKSQVVLTMTNMDPVDTATYYCARHYYYGGSYAMDYWGQGTMVTVSS

SEQ ID NO: 16 - protein, 241 aa, Artificial Sequence
  hCD19-3
QVTLKESGPVLVKPTETLTLTCTVSGVSLPDYGVSWIRQPPGKALEWLAVIWGSETTYYNSALKSRLTISKDTSKSQVVLTMTNMDPVDTATYYCAKHYYYGGSYAMDYWGQGTMVTVSSGGGGSGGGGSGGGGSDIQMTQSPSSVSASVGDRVTITCRASQDISKYLNWYQQKPGKAPKLLIYHTSRLHSGVPSRFSGSGSGTDFTLTISSLQPEDFATYYCQQGNTLPYTFGQGTRLEI

SEQ ID NO: 17 - protein, 241 aa, Artificial Sequence
  hCD19-4
DIQMTQSPSSVSASVGDRVTITCRASQDISKYLNWYQQKPGKAPKLLIYHTSRLHSGVPSRFSGSGSGTDFTLTISSLQPEDFATYYCQQGNTLPYTFGQGTRLEIGGGGSGGGGSGGGGSQVTLKESGPVLVKPTETLTLTCTVSGVSLPDYGVSWIRQPPGKALEWLAVIWGSETTYYNSALKSRLTISKDTSKSQVVLTMTNMDPVDTATYYCAKHYYYGGSYAMDYWGQGTMVTVSS

SEQ ID NO: 18 - DNA, 723 bp, Artificial Sequence
  hCD19-1 scFv
caggtaaccttgaaagaatcaggcccagtcctggtaaagccgacggagacattgacgctgacctgcacagtcagtggagtgtcattgcccgattatggtgtctcttggattcgccagcctcctggcaaggctcttgaatggttggctgtcatctggggcagtgagactacttattataacagtgcgttgaaatcccggctgactattagtaaagatacttccaaaagccaagtagtcctcactatgacaaacatggatcctgtagacacggctacttattattgtgcacggcattactactacggcggctcttatgctatggattactggggtcaaggaacaatggttactgtttcatctggcggcggggggtcaggtgggggtggttccgggggtggtggtagtgacatccaaatgacacaatctccgagtagtctctccgcaagtgtgggcgatcgagttacaattacttgtcgagcctcacaggacatatctaagtacttgaattggtaccaacagaagccggggaaggcaccaaagttgctgctgtatcacactagtagattgcatagtggggtgccctcaagattcagtggaagtggatcaggcactgattacactttgactattagtagtctgcaacaggaagacttcgcaacgtactattgtcagcaaggtaacaccttgccgtacacattcggtcaaggtaccaaagtggaaatt

SEQ ID NO: 19 - DNA, 723 bp, Artificial Sequence
  hCD19-2 scFv
gatattcagatgacccaaagtccgtctagcttgtcagcctccgtaggggacagagttacgataacttgtagagcttcacaggatatatcaaagtaccttaattggtatcaacaaaagcctggtaaagccccgaagcttctcctttaccatacgtctcgactccactccggtgtcccttctcgcttcagtggttctgggagtgggaccgactatacattgaccatctcttctttgcaacaagaggacttcgctacgtattactgtcagcaaggtaatactcttccgtatactttcggacaaggcacgaaagtagaaattggtggtggcggttcaggcgggggtggaagcggggggggtgggtctcaggtcaccttgaaggaatctggacctgttctggtgaagcctacagaaactttgacactgacatgcaccgtctctggtgtatcattgccggactacggtgtgtcatggattaggcaaccgcctggaaaagccttggaatggctcgccgtcatctgggggtctgagacgacctattacaactcagcgttgaagtcacgcctcacgatttcaaaggacacgtctaaatcacaagtggttttgacaatgactaacatggacccggtcgatacggctacttactactgcgctcgccactattattatggggggtcctatgcgatggactattggggacagggcacaatggtgacagtctcaagc

SEQ ID NO: 20 - DNA, 723 bp, Artificial Sequence
  hCD19-3 scFv
caggtgacattgaaagagtccggacctgttctcgtcaaaccgacggagacgctcacccttacatgcacggtcagtggcgtctcactgccagattatggggtttcttggataagacagccccctggcaaggcccttgaatggcttgcggtcatctggggttctgaaacaacatattataacagtgcactcaaatcacgccttacgatttcaaaggatacaagtaaatctcaagttgttctgaccatgacgaatatggatccagtggacaccgcgacgtactattgcgcgaaacattactactacggcggaagctacgctatggactattggggacaaggaacgatggtcactgtctctagcggcgggggtggaagtggaggagggggatcagggggaggaggaagtgacattcaaatgacgcagtctcctagttcagtctctgcatctgtgggcgacagagtcacgattacctgtagggcaagccaggatatctctaagtaccttaattggtatcaacaaaaaccagggaaggccccgaagcttcttatctaccatacaagccgactccatagtggggttccctcacgatttagcgggtctggctctggtacagacttcactctcactattagtagcctgcaaccagaggacttcgcaacctactactgccagcagggaaacactcttccatatacattcggacagggaacccggctcgagatt

SEQ ID NO: 21 - DNA, 723 bp, Artificial Sequence
  hCD19-4 scFv
gatatacaaatgacgcagtctccatcatccgtatccgccagcgtaggagatagagtcaccatcacatgtcgcgcgagtcaggacatcagcaaatacctcaattggtaccagcaaaagcccggcaaggctccaaaattgctgatataccatacgagtaggttgcacagtggagtacccagtagattttctgggtcagggagtggcacagatttcacgctgacaataagtagtcttcaacctgaagatttcgcaacctattactgtcaacaaggtaatacgctgccgtacacgtttggtcagggtacgaggctcgaaatcggtggcgggggaagtggaggtggtggatctgggggtgggggctctcaagttactttgaaagagagtggaccagtgctcgtaaagcctacggagacgcttacccttacgtgcacagtatctggggtgagtctcccagattacggtgtaagttggatacgccagccgccggggaaagccttggaatggctcgcagtgatttggggatccgaaactacctattacaatagcgcccttaaatccaggctgactatttccaaagacacgagcaaatcccaggtcgtccttaccatgacaaacatggacccagttgacaccgctacctactactgtgcgaaacattattactacggtgggagctatgctatggactactggggccaaggcacaatggtcactgttagtagc

SEQ ID NO: 22 - protein, 18 aa, Artificial Sequence
  linker
GSTSGSGKPGSGEGSTKG

SEQ ID NO: 23 - protein, 15 aa, Artificial Sequence
  linker
GGGGSGGGGSGGGGS

SEQ ID NO: 24 - protein, 25 aa, Artificial Sequence
  CD8alpha transmembrane domain
IYIWAPLAGTCGVLLLSLVITLYCK

SEQ ID NO: 25 - DNA, 75 bp, Artificial Sequence
  CD8alpha transmembrane domain
atctacatctgggcgcccttggccgggacttgtggggtccttctcctgtcactggttatcaccctttactgcaaa

SEQ ID NO: 26 - protein, 27 aa, Artificial Sequence
  CD28 transmembrane domain
FWVLVVVGGVLACYSLLVTVAFIIFWV

SEQ ID NO: 27 - DNA, 81 bp, Artificial Sequence
  CD28 transmembrane domain
ttttgggtcctcgtcgtagttggaggggtacttgcctgttatagcctcctggttaccgtagcatttattatattctgggtg

SEQ ID NO: 28 - protein, 41 aa, Artificial Sequence
  CD28 costimulatory domain
RSKRSRLLHSDYMNMTPRRPGPTRKHYQPYAPPRDFAAYRS

SEQ ID NO: 29 - DNA, 123 bp, Artificial Sequence
  CD28 costimulatory domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 30 - protein, 40 aa, Artificial Sequence
  4-1BB costimulatory domain
RGRKKLLYIFKQPFMRPVQTTQEEDGCSCRFPEEEEGGCE

SEQ ID NO: 31 - DNA, 120 bp, Artificial Sequenc
  4-1BB costimulatory domain
cggggcagaaagaaactcctgtatatattcaaacaaccatttatgagaccagtacaaactactcaagaggaagatggctgtagctgccgatttccagaagaagaagaaggaggatgtgaa

SEQ ID NO: 32 - protein, 113 aa, Artificial Sequence
  CD3zeta signaling domain
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 339 bp, Artificial Sequence
  CD3zeta signaling domain
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 114 aa, Artificial Sequence
  CD3zeta signaling domain
LRVKFSRSADAPAYQQGQNQLFNELNLGRREEFDVLDKRRGRDPEMGGKPQRRKNPQEGLYNELQKDKMAEAYSEIGMKGERRRGKGHDGLFQGLSTATKDTFDALHMQALPPR

SEQ ID NO: 35 - DNA, 342 bp, Artificial Sequence
  CD3zeta signaling domain
ctgagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggccttttccagggtctcagtacagccaccaaggacacctttgacgcccttcacatgcaggccctgccccctcgc

SEQ ID NO: 36 - protein, 20 aa, Artificial Sequence
  B2M signal peptide
MSRSVALAVLALLSLSGLEA

SEQ ID NO: 37 - DNA, 60 bp, Artificial Sequence
  B2M signal peptide
atgtcccgctctgttgctttggctgtgctggcccttttgtcccttagcggactggaggcc

SEQ ID NO: 38 - protein, 21 aa, Artificial Sequence
  CD8alpha signal peptide
MALPVTALLLPLALLLHAARP

SEQ ID NO: 39 - DNA, 63 bp, Artificial Sequence
  CD8alpha signal peptide
atggccttaccagtgaccgccttgctcctgccgctggccttgctgctccacgccgccaggccg

SEQ ID NO: 40 - protein, 45 aa, Artificial Sequence
  CD8alpha hinge region
TTTPAPRPPTPAPTIASQPLSLRPEACRPAAGGAVHTRGLDFACD

SEQ ID NO: 41 - DNA, 135 bp, Artificial Sequence
  CD8alpha hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 42 - protein, 39 aa, Artificial Sequence
  CD28 hinge region
IEVMYPPPYLDNEKSNGTIIHVKGKHLCPSPLFPGPSKP

SEQ ID NO: 43 - DNA, 117 bp, Artificial Sequence
  CD28 hinge region
attgaagttatgtatcctcctccttacctagacaatgagaagagcaatggaaccattatccatgtgaaagggaaacacctttgtccaagtcccctatttcccggaccttctaagccc

SEQ ID NO: 44 - protein, 12 aa, Artificial Sequence
  IgG4 hinge region
ESKYGPPCPPCP

SEQ ID NO: 45 - DNA, 36 bp, Artificial Sequence
  IgG4 hinge region
gaaagcaaatacgggccgccgtgtccaccctgtccg

(19) United States
(12) Patent Application Publication
(10) Pub. No.: US 2024/0018236 A1
(43) Pub. Date: Jan. 18, 2024

(54) CD19-TARGETING HUMANIZED ANTIBODY AND USE THEREOF

(71) Applicant: BIOHENG THERAPEUTICS LIMITED, GRAND CAYMAN (KY)

(72) Inventors: Yali ZHOU, NANJING (CN); Xiaoyan JIANG, NANJING (CN); Gong CHEN, NANJING (CN); Jiangtao REN, NANJING (CN); Xiaohong HE, Jiangbei New District, NANJING (CN); Yanbin WANG, Jiangbei New District, NANJING (CN); Lu HAN, NANJING (CN)

(73) Assignee: BIOHENG THERAPEUTICS LIMITED, GRAND CAYMAN (KY)

(21) Appl. No.: 18/255,049

(22) PCT Filed: Dec. 7, 2021
(86) PCT No.: PCT/CN2021/136012

§ 371 Date: May 30, 2023

(30) Foreign Application Priority Data
Dec. 8, 2020 (CN) 202011421860.4

(51) Int. Cl. C07K 16/28; A61K 39/00; G01N 33/574; C07K 16/30

(52) U.S. Cl. CPC C07K 16/2803; A61K 39/4631; A61K 39/464412; G01N 33/57492; A61K 39/4611; C07K 16/3061; C07K 2317/24; G01N 2333/70503; C07K 2317/622

(57) ABSTRACT

The present invention provides a CD19-targeting humanized antibody, and a multispecific antibody, chimeric receptor, antibody conjugate, pharmaceutical composition, and kit comprising the CD19-targeting humanized antibody, and a use thereof in the diagnosis/treatment/prevention of diseases associated with CD19 expression.

### TECHNICAL FIELD

The present disclosure belongs to the field of immunotherapy. More specifically, the present disclosure relates to a CD19-targeting humanized antibody and use thereof in the prevention and/or treatment and/or diagnosis of diseases.

### BACKGROUND ART

CD19, a type I transmembrane protein, consisting of 556 amino acids, belongs to the immunoglobulin superfamily and is expressed on the surface of B lymphocytes and follicular dendritic cells. CD19 forms a receptor complex with CD21, CD81 and CD225 on a B cell to regulate the development, proliferation and differentiation of the B cell, in which CD19 plays a major role in signal transduction. CD19 is only expressed in normal and malignant B cells, almost not expressed in other tissues, and CD19 is not lost during the malignant transformation of B cells, and is still effective for refractory/relapsed cases, making CD19 an effective target for the diagnosis and treatment of B cell malignancies. At present, both ADC and CAR-T cell therapy targeting CD19 have shown good therapeutic effects against B cell malignancies in clinical practice.

The present disclosure aims to provide a CD19-targeting humanized antibody and use thereof in the prevention and/or treatment and/or diagnosis of diseases.

### SUMMARY

In a first aspect, the present disclosure provides a CD19-targeting humanized antibody, comprising a light chain variable region and a heavy chain variable region, wherein the light chain variable region comprises CDR-L1 as set forth in SEQ ID NO: 1, CDR-L2 as set forth in SEQ ID NO: 2, and CDR-L3 as set forth in SEQ ID NO: 3, and the heavy chain variable region comprises CDR-H1 as set forth in SEQ ID NO: 4, CDR-H2 as set forth in SEQ ID NO: 5, and CDR-H3 as set forth in SEQ ID NO: 6, and the light chain variable region has at least 90% identity to the amino acid sequence as set forth in SEQ ID NO: 10 or 12, and the heavy chain variable region has at least 90% identity to the amino acid sequence as set forth in SEQ ID NO: 11 or 13.

In an embodiment, the anti-CD19 humanized antibody comprises a light chain variable region selected from the group consisting of SEQ ID NOs: 10 and 12, and a heavy chain variable region selected from the group consisting of SEQ ID NOs: 11 and 13.

In an embodiment, the anti-CD19 humanized antibody comprises a linker selected from the group consisting of SEQ ID NOs: 22 and 23.

In an embodiment, the anti-CD19 humanized antibody has an amino acid sequence selected from the group consisting of SEQ ID NOs: 14-17.

The present disclosure further provides a nucleic acid molecule encoding the anti-CD19 humanized antibody as described above. Therefore, in an embodiment, the nucleic acid molecule encoding the anti-CD19 humanized antibody has at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NO: 18-21, and the anti-CD19 humanized antibody encoded thereby can specifically bind to the CD19 antigen. Preferably, the nucleic acid molecule encoding the anti-CD19 humanized antibody is selected from SEQ ID NO: 18-21.

In another aspect, the present disclosure further provides a multispecific antibody (preferably bispecific antibody or trispecific antibody), which comprises the anti-CD19 humanized antibody as described above, and one or more second antibodies or antigen-binding portions thereof that specifically bind to antigens different from CD19.

In an embodiment, the second antibody or antigen-binding portion thereof may be in the form of any antibody or antibody fragment, such as a full-length antibody, Fab, Fab′, (Fab′)₂, Fv, scFv, scFv-scFv, a minibody, a diabody or sdAb.

The present disclosure further provides a vector comprising a nucleic acid molecule encoding the anti-CD19 humanized antibody or the multispecific antibody as described above, and a host cell expressing the anti-CD19 humanized antibody or the multispecific antibody.

In another aspect, the present disclosure further provides a chimeric antigen receptor, which comprises the anti-CD19 humanized antibody of the present disclosure, a transmembrane domain and an intracellular signaling domain. Preferably, the chimeric antigen receptor further comprises one or more co-stimulatory domains. In an embodiment, the chimeric antigen receptor of the present disclosure may further comprises a second antibody or antigen-binding portion thereof that targets a tumor antigen different from CD19.

The present disclosure further provides a nucleic acid molecule encoding the chimeric antigen receptor targeting CD19 as defined above, and a vector comprising the nucleic acid molecule.

The present disclosure further provides a cell, preferably an immune cell, such as a T cell, a NK cell, a NKT cell, a macrophage, a dendritic cell, comprising a chimeric antigen receptor targeting CD19 as defined above.

In another aspect, the present disclosure further provides an antibody conjugate comprising the anti-CD19 humanized antibody defined herein and a second functional structure, wherein the second functional structure is selected from the group consisting of an Fc, a radioisotope, a structure moiety for extending half-life, a detectable marker and a drug.

In an embodiment, the structure moiety for extending half-life is selected from the group consisting of an albumin-binding structure, a transferrin-binding structure, a polyethylene glycol molecule, a recombinant polyethylene glycol molecule, a human serum albumin, a fragment of human serum albumin, and a polypeptide (including an antibody) binding to human serum albumin. In an embodiment, the detectable marker is selected from the group consisting of a fluorophore, a chemiluminescent compound, a bioluminescent compound, an enzyme, an antibiotic resistance gene, and a contrast agent. In an embodiment, the drug is selected from the group consisting of a cytotoxin and an immunomodulator.

In another aspect, the present disclosure further provides a detection kit comprising the humanized antibody, the multispecific antibody, the antibody conjugate, the chimeric antigen receptor or the engineered immune cell described in the present disclosure.

In another aspect, the present disclosure further provides a pharmaceutical composition comprising the humanized antibody, the chimeric antigen receptor, the multispecific antibody, the antibody conjugate, or the engineered immune cell described in the present disclosure, and one or more pharmaceutically acceptable excipients.

In another aspect, the present disclosure further provides a method for treating and/or preventing and/or diagnosing diseases associated with CD19 expression, comprising administering to a subject the humanized antibody, the chimeric antigen receptor, the multispecific antibody, the antibody conjugate, or the pharmaceutical composition as described above.

### BRIEF DESCRIPTION OF DRAWINGS

FIG. 1 shows the expression levels of humanized CD19 scFv in CAR-T cells.

FIG. 2 shows the killing effect of CAR-T cells on Nalm6 target cells at different effector-to-target ratios.

FIG. 3 shows the cytokine release level after co-culture of CAR-T cells and target cells Nalm6 or non-target cells 293F.

### DETAILED DESCRIPTION OF EMBODIMENTS

Unless otherwise specified, all scientific and technical terms used herein have the same meaning as commonly understood by one of ordinary skill in the art to which this disclosure belongs.

Anti-CD19 HUMANIZED ANTIBODY

As used herein, the term “antibody” has the broadest meaning understood by those skilled in the art and includes monoclonal antibodies (including whole antibodies), polyclonal antibodies, multivalent antibodies, multispecific antibodies (e.g., bispecific antibodies), and antibody fragments or synthetic polypeptides carrying one or more CDR sequences capable of exhibiting the desired biological activity. The antibodies of the present disclosure may be of any class (e.g., IgG, IgE, IgM, IgD, IgA, etc.) or subclass (e.g., IgG1, IgG2, IgG2a, IgG3, IgG4, IgA 1, IgA2, etc.).

Typically, whole antibodies comprise two heavy chains and two light chains disulfide-bonded together, each light chain being disulfide-bonded to a respective heavy chain, to form a “Y” configuration. Each heavy chain comprises a heavy chain variable region (VH) and a heavy chain constant region, wherein the heavy chain variable region comprises three complementarity determining regions (CDRs): CDR-H1, CDR-H2 and CDR-H3, and the heavy chain constant region comprises three constant domains: CH1, CH2 and CH3. Each light chain comprises a light chain variable region (VL) and a light chain constant region, wherein the light chain variable region comprises three CDRs: CDR-L1, CDR-L2 and CDR-L3, and the light chain constant region comprises a constant domain CL. In the heavy/light chain variable regions, the CDRs are separated by more conserved framework regions (FRs). The heavy/light chain variable regions are responsible for the recognition and binding of the antigen, while the constant regions mediate the binding of the antibody to host tissues or factors, including various cells of the immune system (e.g., effector cells) and the first component of the classical complement system.

The precise amino acid sequence boundaries for a given CDR or FR can be readily determined using a number of numbering schemes well known in the art, including: Kabat et al. (1991), “Sequences of Proteins of Immunological Interest,” 5th Edition, Public Health Service, National Institutes of Health, Bethesda, MD (“Kabat” numbering scheme); A1-Lazikani et al., (1997) JMB 273, 927-948 (“Chothia” numbering scheme); MacCallum et al., J. Mol. Biol 262:732-745 (1996), “Antibody-antigen interactions: Contact analysis and binding sitetopography,” J. Mol. Biol. 262, 732-745″ (“Contact” numbering scheme); Lefranc MP et al., “IMGT unique numbering for immunoglobulin and T cell receptor variable domains and Ig superfamily V-like domains,” Dev Comp Immunol, 2003 Jan; 27(1):55-77 (“IMGT” numbering scheme); Honegger A and Pltickthun A, “Yet another numbering scheme for immunoglobulin variable domains: an automatic modeling and analysis tool,” JMol Biol, 2001 Jun 8; 309(3):657-70 (“Aho” numbering scheme); and Martin et al., “Modeling antibody hypervariable loops: a combined algorithm,” PNAS, 1989, 86(23):9268-9272 (“AbM” numbering scheme).

The boundaries of a given CDR or FR may vary depending on the scheme used for identification. For example, the Kabat scheme is based on structural alignment, while the Chothia scheme is based on structural information. Both the Kabat and Chothia numbering schemes are based on the sequence length of the most common antibody regions where insertions are provided by caret letters (e.g., “30a”) and deletions occur in some antibodies. These two schemes place certain insertions and deletions (“indels”) at different positions, resulting in different numbering. The Contact scheme is based on the analysis of complex crystal structures and is similar in many respects to the Chothia numbering scheme. The AbM scheme is a compromise between the Kabat and Chothia definitions and is based on the scheme used by the AbM antibody modeling software of Oxford Molecular.

Thus, unless otherwise specified, a “CDR” of a given antibody or region thereof (e.g., variable region thereof) is understood to encompass the CDRs defined by any of the above schemes or other known schemes. For example, where it is specified that a particular CDR (e.g., CDR3) comprises a given amino acid sequence, it is understood that such a CDR may also have the sequence of the corresponding CDR (e.g., CDR3) as defined by any of the above schemes or other known schemes. Likewise, unless otherwise specified, FRs for a given antibody or region thereof (e.g., variable region thereof) are understood to encompass FRs as defined by any of the above schemes or other known schemes. The numbering scheme used to define the boundaries of CDRs and FRs in this application adopts the Chothia scheme.

As used herein, a “humanized” antibody refers to an antibody in which all or substantially all CDR amino acid residues are derived from non-human CDRs and all or substantially all FR amino acid residues are derived from human FRs. “Humanized forms” of non-human antibodies refer to variants of such non-human antibodies that have been humanized to generally reduce immunogenicity in humans, while retaining the specificity and affinity of the parent non-human antibody. In some embodiments, some FR residues in a humanized antibody are substituted with corresponding residues from a non-human antibody (e.g., an antibody from which the CDR residues are derived), e.g., to restore or improve antibody specificity or affinity.

Humanized antibodies and methods for their preparation are well known to those skilled in the art, see e.g. Almagro and Fransson, Front. Biosci. 13:1619-1633 (2008). Human framework regions that can be used for humanization include, but are not limited to: framework regions selected using a “best fit” approach; framework regions of the consensus sequences derived from light or heavy chain variable regions shared by human antibodies of a particular subgroup; human mature (somatically mutated) framework regions or human germline framework regions; and framework regions obtained from screening FR libraries.

As used herein, the term “antibody fragment” or “antigen-binding portion” comprises only a portion of an intact antibody, and typically comprises the antigen-binding site of the intact antibody and thus retains the ability to bind antigen. Examples of antibody fragments of the present disclosure include, but are not limited to: Fab, Fab′, F(ab′)2, Fd fragment, Fd′, Fv fragment, scFv, disulfide-linked Fv (sdFv), antibody heavy chain variable region (VH) or light chain variable region (VL), linear antibody, “diabody” with two antigen binding sites, single domain antibody, nanobody, a natural ligand for the antigen or a functional fragment thereof. Accordingly, an “antibody” of the present disclosure encompasses antibody fragments as defined above.

In an embodiment, the anti-CD19 humanized antibody of the present disclosure is selected from the group consisting of IgG, Fab, Fab′, F(ab′)2, Fv, scFv, Fd, Fd′, sdFv, linear antibody, diabody, single domain antibody and nanobody, preferably scFv. “Single-chain antibody” and “scFv” are used interchangeably herein and refer to an antibody formed by linking the heavy chain variable region (VH) and the light chain variable region (VL) of an antibody through a linker. The optimal length and/or amino acid composition of the linker can be selected. The length of the linker may significantly affect the folding and interaction of the variable domain of scFv. For selection of linker size and composition, see, e.g., Hollinger et al., 1993 Proc Natl Acad. Sci. U.S.A. 90:6444-6448; U.S. Patent Application Publication Nos. 2005/0100543, 2005/0175606, 2007/0014794 and PCT Publication Nos. WO2006/020258 and WO2007/024715, the entire contents of which are incorporated herein by reference. Commonly used linkers are, for example, GSTSGSGKPGSGEGSTKG (SEQ ID NO: 22), GGGGSGGGGSGGGGS (SEQ ID NO: 23). A scFv may comprise VH and VL linked in any order, e.g. VH-linker-VL or VL-linker-VH.

In an embodiment, the present disclosure provides an anti-CD19 humanized antibody, comprising a light chain variable region and a heavy chain variable region, wherein the light chain variable region comprises CDR-L1 as set forth in SEQ ID NO: 1, CDR-L2 as set forth in SEQ ID NO: 2, and CDR-L3 as set forth in SEQ ID NO: 3, and the heavy chain variable region comprises CDR-H1 as set forth in SEQ ID NO: 4, CDR-H2 as set forth in SEQ ID NO: 5, and CDR-H3 as set forth in SEQ ID NO: 6, and the light chain variable region has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 10 and 12, or has one or several (e.g., at most 10, at most 9, at most 8, at most 7, at most 6, at most 5, at most 4, at most 3, at most 2) conservative modifications of amino acids compared to SEQ ID NO: 10 or 12, and the heavy chain variable region has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NOs: 11 and 13, or has one or several (e.g., at most 10, at most 9, at most 8, at most 7, at most 6, at most 5, at most 4, at most 3, at most 2) conservative modifications of amino acids compared to SEQ ID NO: 11 or 13.

In an embodiment, the anti-CD19 humanized antibody comprises a light chain variable region selected from the group consisting of SEQ ID NOs: 10 and 12, and a heavy chain variable region selected from the group consisting of SEQ ID NOs: 11 and 13.

In an embodiment, the anti-CD19 humanized antibody has an amino acid sequence selected from the group consisting of SEQ ID NOs: 14-17.

As used herein, the term “sequence identity” means the degree to which two (nucleotide or amino acid) sequences in alignment have the same residue at the same position, and is usually expressed as a percentage. Preferably, identity is determined over the entire length of the sequences being compared. Therefore, two copies of the exact same sequence have 100% identity. Those skilled in the art know that several algorithms can be used to determine sequence identity, such as Blast (Altschul et al. (1997) Nucleic Acids Res. 25:3389-3402), Blast2 (Altschul et al. (1990) J. Mol. Biol. 215: 403-410), Smith-Waterman (Smith et al. (1981) J. Mol. Biol. 147:195-197) and ClustalW.

As used herein, the term “conservative modification” refers to an amino acid modification that does not significantly affect or alter the binding characteristics of an antibody or antibody fragment comprising the amino acid sequence. These conservative modifications include amino acid substitutions, additions and deletions. Modifications can be introduced into the antibodies of the present disclosure by standard techniques known in the art, such as site-directed mutagenesis and PCR-mediated mutagenesis. A conservative amino acid substitution is one in which an amino acid residue is replaced by an amino acid residue with a similar side chain Families of amino acid residues with similar side chains have been defined in the art and include those with basic side chain (e.g., lysine, arginine, histidine), acidic side chain (e.g., aspartic acid, glutamic acid), uncharged polar side chain (e.g., glycine, asparagine, glutamine, serine, threonine, tyrosine, cysteine), non-polar side chain (e.g., alanine, valine, leucine, isoleucine, proline, phenylalanine, methionine, tryptophan), β-branched side chain (e.g., threonine, valine, isoleucine) and aromatic side chain (e.g., tyrosine, phenylalanine, tryptophan, histidine). Conservative modifications can be selected, for example, on the basis of similarity in polarity, charge, solubility, hydrophobicity, hydrophilicity, and/or the amphipathic nature of the residues involved.

In an aspect, the present disclosure further provides a multispecific antibody (preferably a bispecific antibody or a trispecific antibody) comprising the anti-CD19 humanized antibody as described above, and one or more second antibodies that specifically bind to antigens different from CD19.

As used herein, the term “multispecific” means that the antigen binding protein has polyepitopic specificity (i.e., is capable of specifically binding two, three or more different epitopes on one biomolecule or is capable of specifically binding epitopes on two, three or more different biomolecules). As used herein, the term “bispecific” means that an antigen binding protein has two different antigen binding specificities.

In an embodiment, the second antibody may be in the form of any antibody or antibody fragment, such as a full-length antibody, Fab, Fab′, (Fab′) 2, Fv, scFv, scFv-scFv, a minibody, a diabody or sdAb.

Thus, in an embodiment, the second antibody targets an antigen selected from the group consisting of: BCMA, CD4, CD5, CD7, CD8, CD14, CD15, CD20, CD21, CD22, CD23, CD25, CD33, CD37, CD38, CD40, CD40L, CD46, CD52, CD54, CD194, CD80, CD126, CD138, B7, MUC-1, Ia, HM1.24, HLA-DR, tenascin, angiogenic factor, VEGF, PIGF, ED-B fibronectin, oncogene, oncogene product, CD66a-d, necrosis antigen, Ii, IL-2, T101, TAC, IL-6, ROR1, TRAIL-R1 (DR4), TRAIL-R2 (DR5), tEGFR, Her2, L1-CAM, mesothelin, CEA, hepatitis B surface antigen, antifolate receptor, CD24, CD30, CD44, EGFR, EGP-2, EGP-4, EPHa2, ErbB2, ErbB3, ErbB4, ErbB dimer, EGFR vIII, FBP, FCRL5, FCRH5, fetal acetylcholine receptor, GD2, GD3, G protein-coupled receptor type C family 5D (GPRC5D), HMW-MAA, IL-22R-a, IL-13R-a2, kdr, lc light chain, Lewis Y, L1-cell adhesion molecule (L1-CAM), melanoma-associated antigen (MAGE)-A1, MAGE-A3, MAGE-A6, melanoma preferentially expressed antigen (PRAME), survivin, EGP2, EGP40, TAG72, B7-H6, IL-13 receptor a2 (IL-13Ra2), CA9, CD171, G250/CAIX, HLA-A1, HLA-A2, NY-ESO-1, PSCA, folate receptor-a, CD44v6, CD44v7/8, avb6 integrin, 8H9, NCAM, VEGF receptor, 5T4, fetal AchR, NKG2D ligand, dual antigens, antigens associated with common tags, cancer-testis antigen, MUC1, MUC16, NY-ESO-1, MART-1, gp100, carcinoembryonic antigen, VEGF-R2, carcinoembryonic antigen (CEA), prostate specific antigen, PSMA, Her2/neu, estrogen receptor, progesterone receptor, ephrin B2, CD123, c-Met, GD-2, O-acetylated GD2 (OGD2), CE7, Wilms tumor 1 (WT-1), cyclin A2, CCL-1, hTERT, MDM2, CYP1B, WT1, livin, AFP, p53, cyclin (D1), CS-1, BAFF-R, TACI, CD56, TIM-3, CD123, cell cyclins (e.g., cyclin A1 (CCNA1)) and/or pathogen-specific antigens, biotinylated molecules, molecules expressed by HIV, HCV, HBV and/or other pathogens; and/or neo-epitopes or neoantigens.

Nucleic Acid, Vector, Host Cell

In another aspect, the present disclosure relates to a nucleic acid molecule encoding the anti-CD19 antibody or multispecific antibody of the present disclosure. The nucleic acid of the present disclosure may be RNA, DNA or cDNA. According to an embodiment of the present disclosure, the nucleic acid of the present disclosure is a substantially isolated nucleic acid.

In an embodiment, the nucleic acid molecule encoding the anti-CD19 antibody has at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NO: 18-21, and the anti-CD19 antibody encoded thereby can specifically bind to CD19. Preferably, the nucleic acid molecule encoding the anti-CD19 antibody is as set forth in SEQ ID NOs: 18-21.

The nucleic acid of the present disclosure may also be in the form of a vector, may be present in a vector and/or may be part of a vector, such as a plasmid, cosmid or YAC. The vector may especially be an expression vector, i.e., a vector providing for expression of the anti-CD19 antibody in vitro and/or in vivo (i.e., in a suitable host cell, host organism and/or expression system). The expression vector typically comprises at least one nucleic acid molecule of the present disclosure operably linked to one or more suitable expression regulatory elements (e.g., promoter, enhancer, terminator, etc.). Selection of such regulatory elements and their sequences for expression in a particular host is well known to those skilled in the art. Specific examples of regulatory elements and other elements useful or necessary for expression of the anti-CD19 antibody of the present disclosure include, but are not limited to, promoter, enhancer, terminator, integrator, selectable marker, leader sequence, reporter gene.

In another aspect, the present disclosure further provides a host cell expressing the anti-CD19 antibody, the multispecific antibody of the present disclosure and/or a host cell containing the nucleic acid or vector of the present disclosure. Preferred host cells of the present disclosure are bacterial cells, fungal cells or mammalian cells.

Suitable bacterial cells include cells of Gram-negative bacterial strains (e.g., Escherichia coli strains, Proteus strains, and Pseudomonas strains) and Gram-positive bacterial strains (e.g., Bacillus strains, Streptomyces strains, Staphylococcus strains and Lactococcus strains).

Suitable fungal cells include cells of species of Trichoderma, Neurospora, and Aspergillus; or cells of species of Saccharomyces (e.g., Saccharomyces cerevisiae), Schizosaccharomyces (e.g., Schizosaccharomyces pombe), Pichia (e.g., Pichiapastoris and Pichia methanolica) and Hansenula.

Suitable mammalian cells include, for example, HEK293 cells, CHO cells, BHK cells, HeLa cells, COS cells, and the like.

However, amphibian cells, insect cells, plant cells, and any other cells known in the art for expressing heterologous proteins can also be used in the present disclosure.

Recombinant Receptor

In another aspect, the present disclosure further provides a recombinant receptor, such as a recombinant TCR receptor or a chimeric antigen receptor comprising the anti-CD19 humanized antibody as described above. Preferably, the present disclosure further provides a chimeric antigen receptor comprising the anti-CD19 humanized antibody as described above.

As used herein, the term “chimeric antigen receptor” or “CAR” refers to an artificially constructed hybrid polypeptide that generally includes a ligand-binding domain (e.g., an antigen-binding portion of an antibody), a transmembrane domain, an optional co-stimulatory domain, and an intracellular signaling domain, which domains being connected by linkers. CARs are able to redirect the specificity and reactivity of T cells and other immune cells to a selected target in a non-MHC-restricted manner through the antigen-binding properties of antibodies.

In an embodiment, the present disclosure provides a chimeric antigen receptor comprising the anti-CD19 humanized antibody or the multispecific antibody comprising the anti-CD19 humanized antibody as described above, a transmembrane domain and an intracellular signaling domain.

As used herein, the term “transmembrane domain” refers to a polypeptide structure that enables expression of a chimeric antigen receptor on the surface of an immune cell (e.g., a lymphocyte, an NK cell, or an NKT cell), and guides a cellular response of the immune cell against the target cell. The transmembrane domain may be natural or synthetic, and also may be derived from any membrane-bound protein or transmembrane protein. The transmembrane domain is capable of signaling when the chimeric antigen receptor binds to the target antigen. The transmembrane domains particularly suitable for use in the present disclosure may be derived from, for example, a TCRα chain, a TCRβ chain, a TCRγ chain, a TCRδ chain, a CD3 subunit, a CD3E subunit, a CD3γ subunit, a CD36 subunit, CD45, CD4, CD5, CD8a, CD9, CD16, CD22, CD33, CD28, CD37, CD64, CD80, CD86, CD134, CD137, CD154, and functional fragments thereof. Alternatively, the transmembrane domain may be synthesized and may mainly contain hydrophobic residues such as leucine and valine. Preferably, the transmembrane domain is derived from CD8a chain or CD28, and has at least 70%, preferably at least 80%, more preferably at least 90%, at least 95%, at least 97% or at least 99% or 100% sequence identity to the amino acid sequence as set forth in SEQ ID NO: 24 or 26, or the encoding sequence thereof has at least 70%, preferably at least 80%, more preferably at least 90%, at least 95%, at least 97% or at least 99% or 100% sequence identity to the nucleic acid molecule as set forth in SEQ ID NO: 25 or 27.

As used herein, the term “intracellular signaling domain” refers to a protein portion that transduces an effector function signal and guides a cell to perform a specified function. In an embodiment, the intracellular signaling domain contained in the chimeric antigen receptor of the present disclosure may be intracellular sequences of a T cell receptor and a co-receptor, upon binding of antigen receptor, which act together to initiate signaling, as well as any derivative or variant of these sequences and any synthetic sequence having the same or similar function. The intracellular signaling domain may contain many immunoreceptor tyrosine-based activation motifs (ITAM). Examples of intracellular signaling domain of the present disclosure include, but are not limited to, intracellular regions of FcRy, Fen, CD3γ, CD36, CD3E, CD3, CD22, CD79a, CD79b, and CD66d. In a preferred embodiment, the signaling domain of the CAR of the present disclosure may contain a CD3 intracellular region, which has at least 70%, preferably at least 80%, more preferably at least 90%, at least 95%, at least 97%, or at least 99% or 100% sequence identity to the amino acid sequence as set forth in SEQ ID NO: 32 or 34, or the encoding sequence thereof has at least 70%, preferably at least 80%, more preferably at least 90%, at least 95%, at least 97%, or at least 99% or 100% sequence identity to the nucleic acid molecule as set forth in 33 or 35.

In an embodiment, the chimeric antigen receptors of the present disclosure may further comprise a hinge region located between the antibody and the transmembrane domain. As used herein, the term “hinge region” generally refers to any oligopeptide or polypeptide that functions to link a transmembrane domain to an antibody. Specifically, the hinge region serves to provide greater flexibility and accessibility to the antibody. The hinge region may comprise up to 300 amino acids, preferably 10 to 100 amino acids and most preferably 25 to 50 amino acids. The hinge region may be completely or partially derived from a natural molecule, for example, completely or partially from the extracellular region of CD8, CD4 or CD28, or completely or partially from an antibody constant region. Alternatively, the hinge region may be a synthetic sequence corresponding to a naturally occurring hinge sequence, or may be a completely synthetic hinge sequence. In a preferred embodiment, the hinge region comprises a hinge region portion of CD8a, CD28, an Fc γ RIII a receptor, IgG4, or IgG1, more preferably a hinge from CD8a, CD28 or IgG4, and has at least 70%, preferably at least 80%, more preferably at least 90%, at least 95%, at least 97% or at least 99% or 100% sequence identity to the amino acid sequence as set forth in SEQ ID NO: 40, 42 or 44, or the encoding sequence thereof has at least 70%, preferably at least 80%, more preferably at least 90%, at least 95%, at least 97% or at least 99% or 100% sequence identity to the nucleic acid molecule as set forth in SEQ ID NO: 41, 43 or 45.

In an embodiment, the chimeric antigen receptor may also comprise one or more co-stimulatory domains. The co-stimulatory domain may be an intracellular functional signaling domain from a co-stimulatory molecule, which comprises the entire intracellular portion of the co-stimulatory molecule, or a functional fragment thereof. A “co-stimulatory molecule” refers to a cognate binding partner that specifically binds to a co-stimulatory ligand on a T cell, thereby mediating a co-stimulatory response (e.g., proliferation) of the T cell. co-stimulatory molecules include, but are not limited to, MHC class 1 molecules, BTLA, and Toll ligand receptors. Examples of co-stimulatory domains of the present disclosure include, but are not limited to, co-stimulatory signaling domains derived from the following proteins: TLR1, TLR2, TLR3, TLR4, TLRS, TLR6, TLR7, TLR8, TLR9, TLR10, CARD11, CD2, CD7, CD8, CD18 (LFA-1), CD27, CD28, CD30, CD40, CD54 (ICAM), CD83, CD134 (0X40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM, and ZAP70. Preferably, the co-stimulatory domain of the CAR of the present disclosure is from 4-1BB, CD28 or 4-1BB+CD28. In an embodiment, the 4-1BB co-stimulatory domain has at least 70%, preferably at least 80%, more preferably at least 90%, at least 95%, at least 97% or at least 99% or 100% sequence identity to the amino acid sequence as set forth in SEQ ID NO: 30, or the coding sequence thereof has at least 70%, preferably at least 80%, more preferably at least 90%, at least 95%, at least 97% or at least 99% or 100% sequence identity to the nucleic acid molecule as set forth in SEQ ID NO: 31. In an embodiment, the CD28 co-stimulatory domain has at least 70%, preferably at least 80%, more preferably at least 90%, at least 95%, at least 97% or at least 99% or 100% sequence identity to the amino acid sequence as set forth in SEQ ID NO: 28, or the coding sequence thereof has at least 70%, preferably at least 80%, more preferably at least 90%, at least 95%, at least 97% or at least 99% or 100% sequence identity to the nucleic acid molecule as set forth in SEQ ID NO: 29.

In an embodiment, the CAR of the present disclosure may further comprise a signal peptide such that when it is expressed in a cell such as a T cell, the nascent protein is directed to the endoplasmic reticulum and subsequently to the cell surface. The core of the signal peptide may contain a long hydrophobic amino acid segment, which has a tendency to form a single ct-helix. At the end of the signal peptide, there is usually an amino acid segment capable of being recognized and cleaved by signal peptidase. The signal peptidase may cleave during or after translocation, so as to generate free signal peptide and mature protein. Then, the free signal peptide is digested by a specific protease. Signal peptides that may be used in the present disclosure are well known to those skilled in the art, for example, signal peptides derived from B2M, CD8a, IgG1, GM-CSFRa, and the like. In an embodiment, the signal peptide that can be used in the present disclosure is from B2M or CD8a, and has at least 70%, preferably at least 80%, more preferably at least 90%, at least 95%, at least 97% or at least 99% or 100% sequence identity to the amino acid sequence as set forth in SEQ ID NO: 36 or 38, or the coding sequence thereof has at least 70%, preferably at least 80%, more preferably at least 90%, at least 95%, at least 97% or at least 99% or 100% sequence identity to the nucleic acid molecule as set forth in SEQ ID NO: 37 or 39.

In an embodiment, the CAR comprises the anti-CD19 humanized antibody or the multispecific antibody comprising the anti-CD19 humanized antibody as provided herein, a CD28 or CD8a transmembrane region, a CD28 and/or 4-1BB co-stimulatory domain, and a CD3 intracellular signaling domain. In this embodiment, the CAR may further comprise a signal peptide from B2M, CD8a, IgG1 or GM-CSFRa.

In an embodiment, the CAR may further comprise a second antibody or antigen-binding portion thereof that targets other tumor antigens. Examples of such other tumor antigens include, but are not limited to: BCMA, CD4, CD5, CD7, CD8, CD14, CD15, CD20, CD21, CD22, CD23, CD25, CD33, CD37, CD38, CD40, CD40L, CD46, CD52, CD54, CD194, CD80, CD126, CD138, B7, MUC-1, Ia, HM1.24, HLA-DR, tenascin, angiogenic factor, VEGF, PIGF, ED-B fibronectin, oncogene, oncogene product, CD66a-d, necrosis antigen, Ii, IL-2, T101, TAC, IL-6, ROR1, TRAIL-R1 (DR4), TRAIL-R2 (DRS), tEGFR, Her2, L1-CAM, mesothelin, CEA, hepatitis B surface antigen, antifolate receptor, CD24, CD30, CD44, EGFR, EGP-2, EGP-4, EPHa2, ErbB2, ErbB3, ErbB4, ErbB dimer, EGFR vIII, 1-BP, FCRLS, FCRHS, fetal acetylcholine receptor, GD2, GD3, G protein-coupled receptor type C family 5D (GPRCSD), HMW-MAA, IL-22R-α, IL-13R-α2, kdr, κ light chain, Lewis Y, L1-cell adhesion molecule (L1-CAM), melanoma-associated antigen (MAGE)-A1, MAGE-A3, MAGE-A6, melanoma preferentially expressed antigen (PRAME), survivin, EGP2, EGP40, TAG72, B7-H6, IL-13 receptor a2 (IL-13Ra2), CA9, CD171, G250/CAIX, HLA-A1, HLA-A2, NY-ESO-1, PSCA, folate receptor-a, CD44v6, CD44v7/8, avb6 integrin, 8H9, NCAM, VEGF receptor, 5T4, fetal AchR, NKG2D ligand, dual antigens, antigens associated with common tags, cancer-testis antigen, MUC1, MUC16, NY-ESO-1, MART-1, gp100, carcinoembryonic antigen, VEGF-R2, carcinoembryonic antigen (CEA), prostate specific antigen, PSMA, Her2/neu, estrogen receptor, progesterone receptor, ephrin B2, CD123, c-Met, GD-2, O-acetylated GD2 (OGD2), CE7, Wilms tumor 1 (WT-1), cyclin A2, CCL-1, hTERT, MDM2, CYP1B, WT1, livin, AFP, p53, cyclin (D1), CS-1, BAFF-R, TACI, CD56, TIM-3, CD123, cell cyclins (e.g., cyclin A1 (CCNA1)) and/or pathogen-specific antigens, biotinylated molecules, molecules expressed by HIV, HCV, HBV and/or other pathogens; and/or neo-epitopes or neoantigens.

The present disclosure further provides a nucleic acid molecule encoding the chimeric antigen receptor targeting CD19 as defined above, and a vector comprising the nucleic acid molecule.

As used herein, the term “vector” is an intermediary nucleic acid molecule used to transfer (exogenous) genetic material into a host cell, and in the host cell the nucleic acid molecule can be, for example, replicated and/or expressed. The vector generally includes targeting vectors and expression vectors. The “targeting vector” is a medium that delivers an isolated nucleic acid to the interior of a cell by, for example, homologous recombination or by using a hybridization recombinase specifically targeting a sequence at a site. The “expression vector” is a vector used for transcription of heterologous nucleic acid sequences (for example, those sequences encoding the chimeric antigen receptor polypeptides of the present disclosure) in suitable host cells and the translation of their mRNAs. Suitable vectors that can be used in the present disclosure are known in the art, and many are commercially available. In an embodiment, the vector of the present disclosure includes, but is not limited to, plasmid, virus (e.g., retrovirus, lentivirus, adenovirus, vaccinia virus, Rous sarcoma virus (RSV), polyoma virus, and adeno-associated virus (AAV), etc.), phage, phagemid, cosmid, and artificial chromosome (including BAC and YAC). The vector itself is usually a nucleic acid molecule, and usually is a DNA sequence containing an insert (transgene) and a larger sequence as “backbone” of the vector. Engineered vector typically also comprises an origin of autonomous replication in the host cell (if stable expression of polynucleotide is desired), a selectable marker, and a restriction enzyme cleavage site (e.g., a multiple cloning site, MCS). The vectors may additionally contain elements such as a promoter, a poly-A tail (polyA), 3′ UTR, an enhancer, a terminator, an insulator, an operon, a selectable marker, a reporter gene, a targeting sequence, and/or a protein purification tag. In a specific embodiment, the vector is an in vitro transcription vector.

Engineered Immune Cells

In an aspect, the present disclosure further provides an engineered immune cell expressing the recombinant receptors (such as TCR or CAR) of the present disclosure.

As used herein, the term “immune cell” refers to any cell of the immune system that has one or more effector functions (e.g., cytotoxic cell killing activity, secretion of cytokines, induction of ADCC and/or CDC). For example, the immune cell may be a T cell, a macrophage, a dendritic cell, a monocyte, an NK cell, and/or an NKT cell. In an embodiment, the immune cell is derived from a stem cell, such as an adult stem cell, an embryonic stem cell, a cord blood stem cell, a progenitor cell, a bone marrow stem cell, an induced pluripotent stem cell, a totipotent stem cell, or a hematopoietic stem cell, and so on. Preferably, the immune cell is a T cell. The T cell may be any T cell, such as in vitro cultured T cell, for example, primary T cell, or T cell from in vitro cultured T cell line, e.g., Jurkat, SupT1, etc., or T cell obtained from a subject. Examples of subject include humans, dogs, cats, mice, rats, and transgenic species thereof. The T cell may be obtained from a variety of sources, including peripheral blood monocytes, bone marrow, lymph node tissue, umbilical blood, thymus tissue, tissue from sites of infection, ascites, pleural effusion, spleen tissue, and tumors. The T cell also may be concentrated or purified. The T cell may be at any stage of development including, but not limited to, a CD4+/CD8+ T cell, a CD4+ helper T cell (e.g., Th1 and Th2 cells), CD8+ T cell (e.g., cytotoxic T cell), tumor infiltrating cell, memory T cell, naive T cell, γ δ-T cell, α β-T cell. The T cell may also be autologous or allogeneic T cell. In a preferred embodiment, the immune cell is a human T cell. The T cell may be isolated from the blood of a subject using a variety of techniques known to those of skill in the art, such as Ficoll Immune cells may be derived from the patient to be treated (i.e., autologous cells) or from a healthy donor (i.e., allogeneic cells).

The nucleic acid sequence encoding the chimeric antigen receptor can be introduced into an immune cell using conventional methods known in the art (e.g., by transduction, transfection, transformation). “Transfection” is a process of introducing a nucleic acid molecule or polynucleotide (including a vector) into a target cell. An example is RNA transfection, i.e., the process of introducing RNA (e.g., in vitro transcribed RNA, ivtRNA) into a host cell. This term is mainly used for a non-viral method in eukaryotic cells. The term “transduction” is generally used to describe virus-mediated transfer of nucleic acid molecules or polynucleotides. Transfection of animal cells typically involves opening transient pores or “holes” in the cell membrane, so as to allow uptake of material. Transfection may be carried out using calcium phosphate, by electroporation, by extrusion of cells, or by mixing cationic lipids with the material so as to produce liposomes which fuse with the cell membrane and deposit their cargo into the interior. Exemplary techniques for transfecting eukaryotic host cells include lipid vesicle-mediated uptake, heat shock-mediated uptake, calcium phosphate-mediated transfection (calcium phosphate/DNA co-precipitation), microinjection, and electroporation. The term “transformation” is used to describe the non-virus transfer of a nucleic acid molecule or polynucleotide (including a vector) to bacteria, and also to non-animal eukaryotic cells (including plant cells). Thus, the transformation is a genetic alteration of bacterial or non-animal eukaryotic cells, which is produced by direct uptake of a cell membrane from its surroundings and subsequent incorporation of exogenous genetic material (nucleic acid molecule). The transformation may be achieved by artificial means. In order for transformation to occur, the cell or bacterium must be in a competent state. For prokaryotic transformation, the techniques may include heat shock-mediated uptake, fusion to bacterial protoplasts of intact cells, microinjection, and electroporation. After the nucleic acid or vector is introduced into the immune cells, those skilled in the art may amplify and activate the obtained immune cells by conventional techniques.

In an embodiment, in order to reduce the risk of graft-versus-host disease, the engineered immune cell further comprises suppressed or silenced expression of at least one gene selected from the group consisting of: CD52, GR, dCK, TCR/CD3 genes (e.g., TRAC, TRBC, CD3γ, CD3δ, CD3ε, CD3ξ), MHC related genes (HLA-A, HLA-B, HLA-C, B2M, HLA-DPA, HLA-DQ, HLA-DRA, TAP1, TAP2, LMP2, LMP7, RFX5, RFXAP, RFXANK, CIITA) and immune checkpoint genes such as PD1, LAG3, TIM3, CTLA4, PPP2CA, PPP2CB, PTPN6, PTPN22, PDCD1, HAVCR2, BTLA, CD160, TIGIT, CD96, CRTAM, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT, FOXP3, PRDM1, BATF, GUCY1A2, GUCY1A3, GUCY1B2 and GUCY1B3. Preferably, the engineered immune cell further comprises suppressed or silenced expression of at least one gene selected from the group consisting of: TRAC, TRBC, HLA-A, HLA-B, HLA-C, B2M, RFX5, RFXAP, RFXANK, CIITA, PD1, LAG3, TIM3, CTLA4, more preferably TRAC, TRBC, HLA-A, HLA-B, HLA-C, B2M, RFX5, RFXAP, RFXANK, CIITA.

Methods of inhibiting gene expression or silencing genes are well known to those skilled in the art. For example, antisense RNA, RNA decoys, RNA aptamers, siRNA, shRNA/miRNA, transdominant negative protein (TNP), chimeric/antibody conjugates, chemokine ligands, anti-infective cellular proteins, intracellular antibodies (sFv), nucleoside analogs (NRTI), non-nucleoside analogs (NNRTI), integrase inhibitors (oligonucleotides, dinucleotides, and chemical agents), and protease inhibitors may be used to inhibit the expression of genes. Alternatively, genes can also be silenced by DNA breakage mediated by for example meganucleases, zinc finger nucleases, TALE nucleases or Cas enzymes in CRISPR systems.

In an embodiment, a plurality of immune cells are provided, each immune cell engineered to express one or more chimeric antigen receptors. For example, in some embodiments, an immune cell is engineered to express a chimeric antigen receptor that binds and/or targets CD19 (e.g., a CAR comprising the anti-CD19 humanized antibody of the present disclosure), and another cell is engineered to express a chimeric antigen receptor that binds and/or targets antigens different from CD19. In an embodiment, immune cells may also express multispecific chimeric antigen receptors that target one or more antigens, including CD19. For example, such a multispecific chimeric antigen receptor may comprise a multispecific antibody targeting CD19, or comprise both the anti-CD19 humanized antibody of the present disclosure and antibodies targeting antigens different from CD19. In such embodiments, the plurality of engineered immune cells may be administered together or separately. In an embodiment, the plurality of immune cells may be in the same composition or in different compositions. Exemplary compositions of cells include those described in the following sections of this application.

Antibody Conjugate

In an aspect, the present disclosure provides an antibody conjugate comprising the anti-CD19 humanized antibody as defined herein and a second functional structure, wherein the second functional structure is selected from the group consisting of an Fc, a radioisotope, a structure moiety for extending half-life, a detectable marker and a drug.

In an embodiment, the present disclosure provides an antibody conjugate comprising the anti-CD19 humanized antibody as defined herein and Fc. As used herein, the term “Fc” is used to define the C-terminal region of an immunoglobulin heavy chain, and includes natural Fc and variant Fc. “Natural Fc” refers to a molecule or sequence comprising a non-antigen-binding fragment, whether monomeric or multimeric, produced by digestion of an intact antibody. The source of immunoglobulin from which natural Fc is produced is preferably of human origin. Natural Fc fragments are composed of monomeric polypeptides that can be linked as dimers or multimers through covalent linkages (e.g., disulfide bonds) and non-covalent linkages. Depending on the class (e.g., IgG, IgA, IgE, IgD, IgM) or subtype (e.g., IgG1, IgG2, IgG3, IgA 1, IgGA2), natural Fc molecules have 1-4 intermolecular disulfide bonds between monomeric subunits. An example of a natural Fc is a disulfide-linked dimer produced by papain digestion of IgG (see Ellison et al. (1982), Nucleic Acids Res. 10:4071-9). The term “natural Fc” as used herein generally refers to monomeric, dimeric and multimeric forms. A “variant Fc” refers to an amino acid sequence that differs from that of a “natural” or “wild-type” Fc by virtue of at least one “amino acid modification” as defined herein, also referred to as an “Fc variant”. Thus, “Fc” also includes single-chain Fc (scFc), i.e., a single-chain Fc consisting of two Fc monomers linked by a polypeptide linker, which is capable of naturally folding into a functional dimeric Fc region. In an embodiment, the Fc is preferably the Fc of a human immunoglobulin, more preferably the Fc of a human IgG1.

In an embodiment, the present disclosure provides an antibody conjugate comprising the anti-CD19 humanized antibody as defined herein and a radioactive isotope. Examples of radioisotopes useful in the present disclosure include, but are not limited to, At²¹¹, I¹³¹, I¹²⁵, γ⁹⁰, Re¹⁸⁶, Re¹⁸⁸, sm¹⁵³, Bi²¹², P³², pb²¹², ^{99m}Tc, ¹²³I, ¹⁸F, and ⁶⁸Ga.

In an embodiment, the present disclosure provides an antibody conjugate comprising the anti-CD19 humanized antibody as defined herein and a structure moiety for extending half-life selected from the group consisting of an albumin-binding structure of, a transferrin-binding structure, a polyethylene glycol molecule, a recombinant polyethylene glycol molecule, a human serum albumin, a fragment of human serum albumin, and a polypeptide binding to human serum albumin (including antibody).

In an embodiment, the present disclosure provides an antibody conjugate comprising the anti-CD19 humanized antibody as defined herein and a detectable marker. The term “detectable marker” means herein a compound that produces a detectable signal. For example, the detectable marker may be an MRI contrast agent, a scintigraphy contrast agent, an X-ray imaging contrast agent, an ultrasound contrast agent, an optical imaging contrast agent. Examples of detectable markers include fluorophores (e.g., fluorescein, Alexa, or cyanine), chemiluminescent compounds (e.g., luminol), bioluminescent compounds (e.g., luciferase or alkaline phosphatase), enzymes (e.g., horseradish peroxidase, glucose-6-phosphatase, (3-galactosidase), antibiotics (e.g., kanamycin, ampicillin, chloramphenicol, tetracycline, etc.) resistance genes, and contrast agents (e.g., nanoparticles or gadolinium). Those skilled in the art can select an appropriate detectable marker according to the detection system used.

In an embodiment, the present disclosure provides an antibody conjugate comprising the anti-CD19 humanized antibody as defined herein and a drug conjugated to the anti-CD19 humanized antibody, such as a cytotoxin or an immunomodulator (i.e., an antibody-drug conjugate). Usually, the drug is covalently linked to the antibody, usually by a linker. In an embodiment, the drug is a cytotoxin. In another embodiment, the drug is an immunomodulator. Examples of cytotoxins include, but are not limited to, methotrexate, aminopterin, 6-mercaptopurine, 6-thioguanine, cytarabine, 5-fluorouracil, dacarbazine, nitrogen mustard, thiotepa, chlorambucil, melphalan, carmustine (BSNU), lomustine (CCNU), 1-methylnitrosourea, cyclophosphamide, nitrogen mustard, busulfan, dibromomannitol, streptozocin, mitomycin, cis-dichlorodiamine platinum (II) (DDP), cisplatin, carboplatin, zorubicin, doxorubicin, detorubicin, carminomicin, idarubicin, epirubicin, mitoxantrone, actinomycin D, bleomycin, calicheamicin, mithramycin, antramycin (AMC), vincristine, vinblastine, paclitaxel, ricin, pseudomonas exotoxin, gemcitabine, cytochalasin B, gramicidin D, ethidium bromide, emetine, etoposide, teniposide, colchicine, mitoxantrone, 1-dehydrotestosterone, glucocorticoid, procaine, tetracaine, lidocaine, propranolol, puromycin, procarbazine, hydroxyurea, asparaginase, corticosteroids, mitotane (0, P′-(DDD)), interferon, and a combination thereof. Examples of immunomodulators include, but are not limited to, ganciclovir, etanercept, tacrolimus, sirolimus, voclosporin, cyclosporine, rapamycin, cyclophosphamide, azathioprine, mycophenolate mofetil, methotrexate, glucocorticoid and analogs thereof, cytokines, stem cell growth factors, lymphotoxins, tumor necrosis factor (TNF), hematopoietic factors, interleukins (e.g., IL-1, IL-2, IL-3, IL-6, IL-10, IL-12, IL-18 and IL-21), colony-stimulating factors (e.g., G-CSF and (GM-CSF), interferons (e.g., interferon-α, interferon-β and interferon-γ), stem cell growth factor designated “Si factor”, erythropoietin and thrombopoietin, or a combination thereof.

Kits and Pharmaceutical Compositions

In another aspect, the present disclosure further provides a detection kit comprising the humanized antibody, the multispecific antibody, the antibody conjugate, the chimeric antigen receptor or the engineered immune cell described in the present disclosure.

In another aspect, the present disclosure further provides a pharmaceutical composition comprising the humanized antibody, the chimeric antigen receptor, the multispecific antibody, the antibody conjugate, or the engineered immune cell described in the present disclosure, and one or more pharmaceutically acceptable excipients.

As used herein, the term “pharmaceutically acceptable excipient” refers to a carrier and/or excipient that is pharmacologically and/or physiologically compatible (i.e., capable of triggering a desired therapeutic effect without causing any undesired local or systemic effects) with the subject and active ingredient, and it is well known in the art (see, e.g., Remington's Pharmaceutical Sciences. Edited by Gennaro AR, 19th ed. Pennsylvania: Mack Publishing Company, 1995). Examples of pharmaceutically acceptable excipient include, but are not limited to, filler, binder, disintegrant, coating agent, adsorbent, anti-adherent, glidant, antioxidant, flavoring agent, colorant, sweetener, solvent, co-solvent, buffer agent, chelating agent, surfactant, diluent, wetting agent, preservative, emulsifier, cladding agent, isotonic agent, absorption delaying agent, stabilizer, and tension regulator. It is known to those skilled in the art to select a suitable excipient to prepare the desired pharmaceutical composition of the present disclosure. Exemplary excipients for use in the pharmaceutical composition of the present disclosure include saline, buffered saline, dextrose, and water. Generally, the selection of a suitable excipient depends, in particular, on the active agent used, the disease to be treated, and the desired dosage form of the pharmaceutical composition.

The pharmaceutical composition according to the present disclosure is suitable for multiple routes of administration. Generally, the administration is parenterally accomplished. Parenteral delivery methods comprise topical, intraarterial, intramuscular, subcutaneous, intramedullary, intrathecal, intraventricular, intravenous, intraperitoneal, intrauterine, intravaginal, sublingual, or intranasal administration.

The pharmaceutical composition according to the present disclosure also may be prepared in various forms, such as solid, liquid, gaseous or lyophilized forms, particularly the pharmaceutical composition can be prepared in the form of ointment, cream, transdermal patch, gel, powder, tablet, solution, aerosol, granule, pill, suspension, emulsion, capsule, syrup, elixir, extract, tincture or liquid extract, or in a form particularly suitable for the desired method of administration. Processes known in the present disclosure for producing a medicine may include, for example, conventional mixing, dissolving, granulating, dragee-making, grinding, emulsifying, encapsulating, embedding or lyophilizing process. The pharmaceutical composition as described herein is generally provided for example in a form of solution, and preferably comprises a pharmaceutically acceptable buffer agent.

The pharmaceutical composition according to the present disclosure further may be administered in combination with one or more other agents suitable for the treatment and/or prophylaxis of diseases to be treated. referred examples of agent suitable for the combination include known anti-cancer medicines such as cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E, vincristine and doxorubicin; peptide cytotoxins, such as ricin, diphtheria toxin, pseudomonas exotoxin A, DNase and RNase; radionuclides such as iodine 131, rhenium 186, indium 111, iridium 90, bismuth 210, bismuth 213, actinides 225 and astatine 213; prodrugs such as antibody-directed enzyme prodrugs; immunostimulatory agents such as platelet factor 4, and melanoma growth stimulating protein; antibodies or fragments thereof, such as anti-CD3 antibodies or fragments thereof, complement activators, heterologous protein domains, homologous protein domains, viral/bacterial protein domains and viral/bacterial peptides. In addition, the pharmaceutical composition of the present disclosure also can be used in combination with one or more other treatment methods, such as chemotherapy and radiotherapy.

Therapeutic/Preventive/Diagnostic Use

In another aspect, the present disclosure further provides a method for treating and/or preventing and/or diagnosing diseases associated with CD19 expression, comprising administering to a subject the humanized antibody, the chimeric antigen receptor, the multispecific antibody, the antibody conjugate, or the pharmaceutical composition as described above.

In an embodiment, the disease associated with CD19 expression is a B-cell lymphoma selected from the group consisting of acute lymphoblastic leukemia (ALL), AIDS-related lymphoma, ALK-positive large B-cell lymphoma, Burkitt's lymphoma, chronic lymphocytic leukemia (CLL), classical Hodgkin lymphoma, diffuse large B-cell lymphoma (DLBCL), follicular lymphoma, intravascular large B-cell lymphoma, large B-cell lymphoma caused by HHV8-related multicentric Castleman disease, lymphomatoid granuloma, lymphoplasmacytic lymphoma, mantle cell lymphoma (MCL), marginal zone B-cell lymphoma (MZL), mucosa-associated lymphoid tissue lymphoma (MALT), nodal marginal zone B-cell lymphoma (NMZL), nodular lymphocyte-predominant Hodgkin lymphoma, non-Hodgkin lymphoma, plasmablastic lymphoma, primary central nervous system lymphoma, primary effusion lymphoma, splenic marginal zone lymphoma (SMZL), and Waldendorf s macroglobulinemia or a combination thereof.

In a preferred embodiment, the B-cell lymphoma is acute lymphoblastic leukemia (ALL), chronic lymphocytic leukemia (CLL), diffuse large B-cell lymphoma (DLBCL), follicular lymphoma, mantle cell lymphoma (MCL), marginal zone B-cell lymphoma (MZL), mucosa-associated lymphoid tissue lymphoma (MALT), and non-Hodgkin lymphoma tumor.

The present disclosure will be described in detail below with reference to the accompanying drawings and examples. It should be noted that those skilled in the art should understand that the drawings and the embodiments of the present disclosure are only for the purpose of illustration, and shall not constitute any limitation to the present disclosure. In the case of no contradiction, the embodiments in the present application and the features in the embodiments can be combined with each other.

### EXAMPLES

### Example 1. Preparation of Anti-CD19 Humanized Antibodies

The humanized antibodies were prepared based on murine FMC63 Clone. FMC63 comprises CDR-L1 as set forth in SEQ ID NO: 1, CDR-L2 as set forth in SEQ ID NO: 2, CDR-L3 as set forth in SEQ ID NO: 3, CDR-H1 as set forth in SEQ ID NO: 4, CDR-H2 as set forth in SEQ ID NO: 5, CDR-H3 as set forth in SEQ ID NO: 6. The sequence of the light chain variable region set forth in SEQ ID NO: 7, the sequence of the heavy chain variable region set forth in SEQ ID NO: 8, and the amino acid sequence of the full-length scFv set forth in SEQ ID NO: 9. The specific method of preparing humanized antibodies is as follows: keeping the CDR region sequences of the original antibody unchanged, selecting different human antibody templates for the heavy chain and light chain according to the results of germline alignment and antibody structure simulation, and performing back mutations in the framework region after humanization, to ensure the affinity and specificity of humanized antibodies. Finally, 2 humanized light chain variable region sequences hVL1 (SEQ ID NO: 10) and hVL2 (SEQ ID NO: 12), and 2 humanized heavy chain variable region sequences hVH1 (SEQ ID NO: 11) and hVH2 (SEQ ID NO: 13) were obtained. The above light chain variable region and heavy chain variable region were combined to obtain 4 single-chain antibodies: hCD19-1 (hVH1-linker-hVL1, with the amino acid sequence as set forth in SEQ ID NO: 14, and nucleotide sequence as set forth in SEQ ID NO: 18), hCD19-2 (hVL1-linker-hVH1, with the amino acid sequence as set forth in SEQ ID NO: 15, and nucleotide sequence as set forth in SEQ ID NO: 19), hCD19-3 (hVH2-linker-hVL2, with the amino acid sequence as set forth in SEQ ID NO: 16, and nucleotide sequence as set forth in SEQ ID NO: 20), and hCD19-4 (hVL2-linker-hVH2, with the amino acid sequence as set forth in SEQ ID NO: 17, and nucleotide sequence as set forth in SEQ ID NO: 21).

### Example 2. Preparation of CAR-T Cells Containing Anti-CD19 Humanized Antibodies

Sequences encoding the following proteins were synthesized and cloned into pLVX vector (Public Protein/Plasmid Library (PPL), Cat. No.: PPL00157-4a): CD8a signal peptide (SEQ ID NO:), anti-CD19 humanized antibody (selected from SEQ ID NO:), CD8a hinge region (SEQ ID NO:), CD8a transmembrane region (SEQ ID NO:), 4-1BB intracellular region (SEQ ID NO:) and CD3 intracellular signaling domain (SEQ ID NO:), and the correct insertion of the target sequence was confirmed by sequencing. The amino acid sequence of the anti-CD19 scFv contained in the hCAR19-1 CAR set forth in SEQ ID NO: 14; the amino acid sequence of the anti-CD19 scFv contained in the hCAR19-2 CAR set forth in SEQ ID NO: 15; the amino acid sequence of the anti-CD19 scFv contained in the hCAR19-3 CAR set forth in SEQ ID NO: 16; the amino acid sequence of the anti-CD19 scFv contained in the hCAR19-4 CAR set forth in SEQ ID NO: 17.

Three ml Opti-MEM (Gibco, Cat. No. 31985-070) was added to a sterile tube to dilute the above plasmid, and then packaging vector psPAX2 (Addgene, Cat. No. 12260) and envelope vector pMD2.G (Addgene, Cat. No. 12259) were added according to the ratio of plasmid: viral packaging vector: viral envelope vector=4:2:1. Then, 120 pl X-treme GENE HP DNA transfection reagent (Roche, Cat. No. 06366236001) was added, mixed immediately, and incubated at room temperature for min, and then the plasmid/vector/transfection reagent mixture was added dropwise to the culture flask containing 293T cells. Viruses were collected at 24 hours and 48 hours, pooled, and ultracentrifuged (25000g, 4° C., 2.5 hours) to obtain concentrated lentivirus.

T cells were activated with DynaBeads CD3/CD28 CTS TM (Gibco, Cat. No. 40203D), and were further cultured for 1 day at 37° C. and 5% CO2. Then, the concentrated lentivirus was added, and after 3 days of continuous culture, CAR T cells expressing different CD19 humanized scFv were obtained. Unmodified wild-type T cells were used as negative controls (NT).

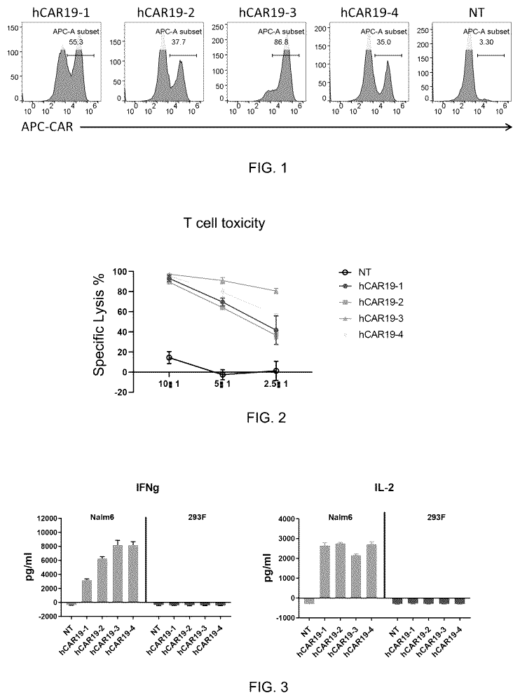

The expression level of scFv on the hCAR19-1 T cells, hCAR19-2 T cells, hCAR19-3 T cells and hCAR19-4 was detected by flow cytometry using Biotin-SP (long spacer) AffiniPure Goat Anti-Mouse IgG, F(ab′) 2 Fragment Specific (min X Hu, Bov, Hrs Sr Prot) (Jackson immunoresearch, Cat. No. 109-065-097) as the primary antibody, APC Streptavidin (BD Pharmingen, Cat. No. 554067) or PE Streptavidin (BD Pharmingen, Cat. No. 554061) as the secondary antibody, and the results are shown in FIG. 1.

FIG. 1 shows the anti-CD19 humanized scFv in the CAR T cells prepared in the present disclosure are effectively expressed.

### Example 3. Killing Effect of CAR T Cells on Target Cells and Release of Cytokines

3.1 The Killing Effect of CAR-T Cells on Target Cells

When T cells kill target cells, the number of target cells will decrease. After co-culturing T cells with target cells expressing luciferase, the number of target cells decreases and the secretion of luciferase decreases accordingly. Luciferase can catalyze the conversion of luciferin into oxidized luciferin, and during this oxidation process, bioluminescence will be generated, and the intensity of this luminescence will depend on the level of luciferase expressed by the target cells. Therefore, the detected fluorescence intensity can reflect the ability of T cells to kill target cells.

In order to detect the killing ability of CAR-T cells on target cells, first Nalm6 target cells carrying the fluorescein gene were plated into a 96-well plate at 1×10⁴/well, and then CAR T cells and NT cells were plated in the 96-well plate with effector-target ratios (i.e., the ratio of effector T cells to target cells) of 10:1, 5:1 and 2.5:1 for co-culture, and the fluorescence value was measured with a microplate reader after 16-18 hours. According to the calculation formula: (average fluorescence value of target cells−average fluorescence value of samples)/average fluorescence value of target cells×100%, the killing efficiency was calculated, and the results are shown in FIG. 2.

FIG. 2 shows, compared with NT, the CAR T cells of the present disclosure specifically kill target cells.

3.2 Cytokine release of CAR-T cells

When T cells kill target cells, the number of target cells decreases and cytokines are released at the same time. According to the following steps, enzyme-linked immunosorbent assay (ELISA) was used to measure the release levels of cytokines IL2 and IFNγ when the CAR T cells of the present disclosure kill target cells.

(1) Collection of cell co-culture supernatant

Target cells were plated in a 96-well plate at a concentration of 1×10⁵/well, and then CAR T and NT cells (negative control) were co-cultured with target cells Nalm6 or non-target cells 293F at a ratio of 1:1, and cell co-culture supernatants were collected after 18-24 hours.

(2) Detection of the secretion of IL-2 and IFN-γ in the supernatant by ELISA

A 96-well plate was coated with Purified anti-human IL2 Antibody (Biolegend, Cat. No. 500302) or Purified anti-human IFN-γ Antibody (Biolegend, Cat. No. 506502) as capture antibody and incubated overnight at 4° C., and then the antibody solution was removed. 250 μL of PBST (1XPBS containing 0.1% Tween) solution containing 2% BSA (sigma, Cat. No. V900933-1 kg) was added, and incubated at 37° C. for 2 hours. The plate was then washed 3 times with 250 μL PBST (1XPBS containing 0.1% Tween). 50 μL of cell co-culture supernatant or standard per well was added and incubated at 37° C. for 1 h, then the plate was washed 3 times with 250 μL of PBST (1XPBS containing 0.1% Tween). Then 50 μL Anti-Interferon gamma antibody [MD-1] (Biotin) (abcam, Cat. No. ab25017) as detection antibody was added to each well, incubated at 37° C. for 1 hour, and the plate was washed 3 times with 250 μL PBST (1XPBS containing 0.1% Tween). Then HRP Streptavidin (Biolegend, Cat. No. 405210) was added, incubated at 37° C. for 30 minutes, and the supernatant was discarded. 250 μL of PBST (1XPBS containing 0.1% Tween) was added for washing 5 times. 50 μL of TMB substrate solution was added to each well. Reactions were allowed to occur at room temperature in the dark for 30 minutes, after which 50 μL of 1 mol/L H₂SO₄ was added to each well to stop the reaction. Within 30 minutes of stopping the reaction, a microplate reader was used to detect the absorbance at 450 nm, and the content of cytokines was calculated according to the standard curve (drawn according to the reading value and concentration of the standard), the results are shown in FIG. 3.

FIG. 3 shows the cytokine release rate of the CAR T cells of the present disclosure is significantly higher than that of the control NT cells.

It should be noted that the above-mentioned are merely for preferred examples of the present disclosure and not used to limit the present disclosure. For one skilled in the art, various modifications and changes may be made to the present disclosure. Those skilled in the art should understand that any amendments, equivalent replacements, improvements, and so on, made within the spirit and principle of the present disclosure, should be covered within the scope of protection of the present disclosure.

1. A CD19-targeting humanized antibody, comprising a light chain variable region and a heavy chain variable region, wherein the light chain variable region comprises CDR-L1 as set forth in SEQ ID NO: 1, CDR-L2 as set forth in SEQ ID NO: 2, and CDR-L3 as set forth in SEQ ID NO: 3, and the heavy chain variable region comprises CDR-H1 as set forth in SEQ ID NO: 4, CDR-H2 as set forth in SEQ ID NO: 5, and CDR-H3 as set forth in SEQ ID NO: 6, and the light chain variable region has at least 90% identity to the amino acid sequence as set forth in SEQ ID NO: 10 or 12, and the heavy chain variable region has at least 90% identity to the amino acid sequence as set forth in SEQ ID NO: 11 or 13.

2. The humanized antibody according to claim 1, wherein the humanized antibody comprises a light chain variable region selected from the group consisting of SEQ ID NOs: 10 and 12, and a heavy chain variable region selected from the group consisting of SEQ ID NOs: 11 and 13.

3. The humanized antibody according to claim 1, wherein the humanized antibody comprises a linker selected from the group consisting of SEQ ID NOs: 22 and 23.

4. The humanized antibody according to claim 1, wherein the humanized antibody has an amino acid sequence selected from the group consisting of SEQ ID NOs: 14-17.

5. A nucleic acid molecule encoding the humanized antibody according to claim 1.

6. A multispecific antibody comprising the humanized antibody according to claim 1 and one or more second antibodies or antigen-binding portions thereof that specifically bind to antigens different from CD19.

7. The multispecific antibody according to claim 5, wherein the one or more second antibodies or antigen binding portions thereof are selected from the group consisting of a full-length antibody, Fab, Fab′, (Fab′)₂, Fv, scFv, scFv-scFv, a minibody, a diabody or sdAb.

8. A vector comprising a nucleic acid molecule encoding the humanized antibody according to claim 1.

9. A host cell expressing the humanized antibody according to claim 1.

10. A chimeric antigen receptor comprising the humanized antibody according to claim 1, a transmembrane domain and intracellular signaling domain.

11. The chimeric antigen receptor according to claim 9, wherein the transmembrane domain is derived from a TCRα chain, a TCRβ chain, a TCRγ chain, a TCRδ chain, a CD3t subunit, a CD3c subunit, a CD3γ subunit, a CD36 subunit, CD45, CD4, CD5, CD8a, CD9, CD16, CD22, CD33, CD28, CD37, CD64, CD80, CD86, CD134, CD137, or CD154.

12. The chimeric antigen receptor according to claim 9, wherein the intracellular signaling domain is selected from the group consisting of intracellular regions of FcRγ, FeRβ, CD3γ, CD3δ, CD3ε, CD3, CD22, CD79a, CD79b, and CD66d.

13. The chimeric antigen receptor according to claim 9, further comprising one or more co-stimulatory domains, wherein the co-stimulatory domain is selected from the group consisting of co-stimulatory signaling domains of TLR1, TLR2, TLR3, TLR4, TLR5, TLR6, TLR7, TLR8, TLR9, TLR10, CARD11, CD2, CD7, CD8, CD18 (LFA-1), CD27, CD28, CD30, CD40, CD54 (ICAM), CD83, CD134 (OX40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM, and ZAP70.

14. An engineered immune cell comprising the chimeric antigen receptor according to claim 10.

15. The engineered immune cell according to claim 14, wherein the engineered immune cell is selected from the group consisting of a T cell, a NK cell, a NKT cell, a macrophage, and a dendritic cell.

16. An antibody conjugate comprising the humanized antibody according to claim 1, and a second functional structure, wherein the second functional structure is selected from the group consisting of an Fc, a radioisotope, a structure moiety for extending half-life, a detectable marker and a drug.

17. The antibody conjugate according to claim 16, wherein the structure moiety for extending half-life is selected from the group consisting of an albumin-binding structure, a transferrin-binding structure, a polyethylene glycol molecule, a recombinant polyethylene glycol molecule, a human serum albumin, a fragment of human serum albumin, and a polypeptide binding to human serum albumin; the detectable marker is selected from the group consisting of a fluorophore, a chemiluminescent compound, a bioluminescent compound, an enzyme, an antibiotic resistance gene, and a contrast agent; and the drug is selected from the group consisting of a cytotoxin and an immunomodulator.

18. A detection kit comprising the humanized antibody according to claim 1, the multispecific antibody according to claim 6 or 7, the chimeric antigen receptor according to claim 10.

19. A pharmaceutical composition comprising the humanized antibody according to claim 1.

20. A method for treating and/or preventing and/or diagnosing a disease associated with CD19 expression, comprising administering to a subject an effective amount of the engineered immune cell according to claim 14.
